(19) United States
(12) Patent Application Publication
(10) Pub. No.: US 2015/0079560 A1
(43) Pub. Date: Mar. 19, 2015

(54) Wearable Monitoring and Training System for Focus and/or Mood

(71) Applicant: Jonathan Daniel Cowan, Goshen, KY (US)

(72) Inventor: Jonathan Daniel Cowan, Goshen, KY (US)

(21) Appl. No.: 14/323,770

(22) Filed: Jul. 3, 2014

Related U.S. Application Data
(60) Provisional application No. 61/842,793, filed on Jul. 3, 2013.

(51) Int. Cl. A61B 5/0482; G06F 3/01; G02B 27/01; G09B 19/00

(52) U.S. Cl. CPC A61B 5/0482; G09B 19/00; G06F 3/015; G06F 3/013; G02B 27/017; G02B 2027/0178
USPC 434/236; 600/545

(57) ABSTRACT

A wearable signal acquisition and computerized display device including at least one first sensor configured to measure at least one of the following attributes from a subject wearing the device: attention, focus, concentration, alertness, and mood. The device has a heads-up display or goggles, glasses, or another head piece having lenses. The measurement is derived from the subject's brainwaves by use of one or more algorithms. The subject wearing the device receives neurofeedback data based on this measurement through the projected image to train them how to control attention, mood, etc. and improve understanding as to how various external stimuli in the real world or virtual world impact these attributes. In one variation the sensors measure brainwave signals at two positions of the subject and use the difference in these measurements to derive an attention indicator signal used to provide neurofeedback.

### CROSS REFERENCE TO RELATED APPLICATIONS

This application claims the priority, benefit and filing date of co-pending U.S. Provisional Application No. 61/842,793 filed Jul. 3, 2013, in the name of the same inventor, Jonathan D. Cowan and having the same title: Wearable Monitoring and Training System for Focus and/or Mood. The entirety of that application is incorporated herein by this reference.

### FIELD OF THE INVENTION

This invention is directed to wearable computing devices, displays, acquisition units, systems, and methods for providing neurofeedback to monitor and improve concentration, alertness, focus, and/or mood.

### BACKGROUND

Unless otherwise indicated herein, the materials described in this section are not prior art to the claims in this application and are not admitted to be prior art by inclusion in this section.

Computing devices such as personal computers, laptop computers, tablet computers, smart phones, cellular phones, and other varieties of electronic devices—and increasingly internet-accessible electronic devices as well—are becoming common to assist with many aspects of contemporary life. In the near future augmented-reality devices that merge computer-generated information with a user's own real-time or live perception of the actual physical world are anticipated to increase in popularity. It would be desirable if these devices not only provided a user with very accessible information about objects and circumstances they encounter in the real world but also with information about how their internal world or psyche, mind, and emotions are processing and responding to external stimuli. In this manner, individuals could train themselves to better deal with the environments and tasks they encounter while receiving valuable personalized data There is a need for a wearable or highly portable interactive device that allows an individual to measure and improve their own mental processes, such as mood, focus and the like. In particular, a device that provides the feedback to an individual undergoing physical therapy that is necessary to enhance the focus of their attention on the muscle groups in question will enhance and speed up the therapeutic process in many individuals.

Wearable devices for monitoring physiologic variables have been used to prevent and treat physical health conditions. Variables that can be monitored include pulse, blood oxygen saturation, blood pressure, temperature, and many more. It would be desirable to also be able to monitor physiologic variables to prevent and treat mental and emotional health conditions. While traditionally, one went to the doctor at regular intervals for a check-up on the status of physiologic variables, it would be preferable to continuously monitor relevant variables without need for frequent doctor visits. It would be desirable if a doctor, therapist, counselor, trainer, coach, or other professional could have continuous access to the streaming results from physiologic monitoring devices along with a patient or user. In this manner, the professional can be alerted if certain threshold criteria are met. Historically, physiologic variables were monitored with medical equipment under the control of a physician, e.g. blood sample, stethoscope, arm band, etc. It would be desirable to have lower profile or less obvious sensors that don't look like medical equipment and blend with an individual's ordinary accessories and apparel such that physiologic variables can be continuously monitored without anyone else knowing.

Recently, wearable computing devices have evolved to include heads-up displays, which may be defined to be electronic devices with screens that provide information to a user wearing the devices while they are going about their business. A heads-up display is positioned near the user's eyes so that a user can see displayed images or information with little or no head movement, making them useful for drivers, pilots, athletes, etc. A computer processing system can be used to generate the images. This can be part of the heads-up equipment or positioned elsewhere and connected to it through wires or wirelessly. Glasses, goggles, headbands, hats, and helmets are examples of objects that can be used to mount heads-up displays.

One potential example of the monitoring of human vital signs is U.S. Pat. No. 6,979,731, teaching a woven or knitted fabric-based sensor with conductive fibers. However, that device does not teach the visual training and feedback methods permitted by the present invention.

Numerous patents relating to the “Google Glass”® product are known as well. In general, while the Google Glass® teaches a wearable visual display, it does not teach toward a brainwave monitoring and tracking system for focus and mood training.

In recent years people have been intrigued by the concept of brainwave actuated phenomena and experimenting with the possibility of brainwave actuated games and devices. This research appears promising not only to improve independence and quality of life for physically handicapped individuals but also as a means for normal individuals and those suffering from mental or emotional vulnerabilities and conditions to control and train their brains and harness the power of their minds. In particular neurofeedback and brainwave feedback rely on the principle of measuring neural activity, then providing this neural activity as a data “input” to the user, who can then adjust their own neural activity in response, which is then measured and the next round of data serves as feedback to the user regarding the effectiveness of the effort. Feedback training can under some circumstances be extremely effective in teaching users to alter their brain activity.

The brain produces electrical signals from at least 0-128 Hertz (“Hz”), which are measurable from the scalp. These signals constitute the electroencephalogram or EEG. According to conventional wisdom signals from about 0-4 Hz often indicate a deep sleep state (the so-called, “delta” range); signals from about 4-8 Hz indicate a reverie or daydreaming state (the so-called, “theta” range); signals from about 8-13 Hz indicate an alert, but less mentally busy state (the so-called, “alpha” range); and, signals above 13 Hz indicate a vigilant state (the so-called, “beta” range).

While this may be true of signals measured from a majority of the cortex, this invention is based on applicant's research, indicating that an additional phenomenon can be reliably demonstrated in the frontal and fronto-central midline portions of the brain surrounding the sites labeled FCz, Fz, AFz and FPz by electroencephalographers. In these areas, which overlie portions of the Executive Attention Network, attention focused on an object causes all of these organized brainwave patterns to diminish in intensity. They are presumably replaced by a higher frequency, very random cortical activation pattern that would be very difficult to measure with a traditional EEG instrument, since it is attenuated by the skull. The present invention monitors focused attention and concentration, by measuring the decrease of organized EEG output from 0-40 Hz at one or more of these sites. The largest decrease in amplitude or power while focusing generally takes place between 0-11 Hz. Applicant's earlier patent, U.S. Pat. No. 5,983,129, set forth a method in which the inhibition of frontal lobe EEG signals is used to provide an attention indicator.

For portability and convenience, it is desirable to integrate a method for detecting this decrease in brainwave output into a computer program which produces output that can be viewed by using a computerized heads-up display. It is even further desirable to provide in a wearable device having a computerized heads-up display a method to detect a signal produced from an individual's brain and to derive therefrom a measure of the individual's intensity of focused attention and/or level of concentration on a particular experience.

The present invention meets these and other needs.

### SUMMARY OF THE INVENTION

According to a first aspect, the invention provides a wearable signal acquisition and computerized display device including at least one first sensor, signal conditioning means and a computerized display in close proximity to a subject's eyes, configured to measure at least one of the following attributes from a subject wearing the device: attention, focus, concentration, alertness, and mood. The wearable signal acquisition and computerized display device may also include a heads-up display. The wearable signal acquisition and computerized display device may also include a projected display created by the device that provides neurofeedback to the subject indicative of performance of one of the measured attributes. Further, the projected display may be interactive with the subject wearing the device in that the projected display is dependent upon at least one of the measured attributes and changes in that attribute.

The wearable signal acquisition and computerized display device may also include goggles or glasses as part of the device. The wearable signal acquisition and computerized display device may be in a form of goggles or glasses. The wearable signal acquisition and computerized display device may be in a form of a helmet, cap, hat, headband, sweatband, or other head gear.

The wearable signal acquisition and computerized display device may further include at least one indicator to provide notification when at least one of the measured attributes meets a threshold performance metric indicating positive or negative performance. The wearable signal acquisition and computerized display device may include at least one additional or second sensor and second signal conditioning means for sensing a physiologic variable from a different position on the subject than the first sensor. The first sensor may be configured to sense a brainwave signal. The first sensor may be configured to sense a brainwave signal near a frontal lobe and the second sensor may be configured to sense a brainwave signal from a more electrically-neutral position. The second sensor may be configured to sense a brainwave signal from an ear.

According to another aspect, the wearable signal acquisition and computerized display device or a computer program cooperating with the device may use an algorithm that takes a difference between the brainwave signals at the first and second sensors to produce a difference brainwave signal and then processes the difference brainwave signal to produce an Attention Indicator signal indicative of the subject's intensity of focused attention. The Attention Indicator signal is inversely proportional to any mathematical transformation of an amplitude measure of the difference brainwave signal. The algorithmic computations, calculations, and processing may be performed within the wearable device itself. The algorithmic computations, calculations, and processing may be performed at another location, either local or remote, via digital or analog communications over wired or wireless connections.

According to another aspect, the present invention provides a method for training an individual to improve at least one of attention, focus, concentration, alertness, and mood, the method including putting on a wearable signal acquisition and computerized display device having a heads-up display and interacting with the wearable signal acquisition and computerized display device to generate neurofeedback data through a projected display visible to the individual. The projected display may be visible on lenses of glasses or goggles of the wearable signal acquisition and computerized display device. The individual may still see and interact with the real physical world while using the wearable signal acquisition and computerized display device and simultaneously viewing the projected display. In one variation of the method, the wearable signal acquisition and computerized display device may be used to teach rehabilitation patients how to focus on a relevant impaired body part to improve body part functionality and quality of treatment while reducing duration of rehabilitation and allowing patients to work on rehabilitation anywhere.

According to still another aspect, the present invention provides a system for remote patient monitoring including the wearable signal acquisition and computerized display device and also including a transmitter for receiving data gathered by the sensor and communicating the data to a monitor of a physician, therapist, counselor, or coach. The monitor may be remote from the wearable signal acquisition and computerized display device and the data may be communicated wirelessly or over a network.

### SUMMARY IN REFERENCE TO CLAIMS

Thus it is one aspect, advantage, objective and embodiment of the present invention to teach a wearable signal acquisition and computerized display device for use in close proximity to a subject's eyes and brain, including a frontal lobe of such subject brain and a more electrically neutral portion of such subject brain, the device comprising:

at least a first sensor capable of producing a first signal;

signal conditioning means;

a computerized display;

the sensor configured to measure at least one attribute from such subject when wearing the device at a first location on such subject, the attribute selected from the group consisting of: attention, focus, concentration, alertness, mood and combinations thereof.

Thus it is a second aspect, advantage, objective and embodiment of the present invention to teach a wearable signal acquisition and computerized display device further comprising a heads-up display.

Thus it is a third aspect, advantage, objective and embodiment of the present invention to teach a wearable signal acquisition and computerized display device further comprising modified glasses as part of the device.

Thus it is another aspect, advantage, objective and embodiment of the present invention to teach a wearable signal acquisition and computerized display device further comprising modified goggles as part of the device.

Thus it is another aspect, advantage, objective and embodiment of the present invention to teach a wearable signal acquisition and computerized display device wherein the device is in a form of a modified item of headgear, the item of headgear selected from the list consisting of: helmet, cap, hat, headband, sweatband, spring-loaded arm mounted on the ear, other head gear and combinations thereof.

Thus it is another aspect, advantage, objective and embodiment of the present invention to teach a wearable signal acquisition and computerized display device further comprising at least one indicator to provide notification when the at least one measured attribute meets a first threshold performance metric, the first threshold performance metric selected from the group consisting of: positive performance, negative performance, time of performance, a performance profile, the integral of performance amplitude over time, and combinations thereof.

Thus it is another aspect, advantage, objective and embodiment of the present invention to teach a wearable signal acquisition and computerized display device further comprising a projected display created by the device that provides neurofeedback to the subject indicative of performance—instantaneous or integrated over a time period—of the at least one measured attribute.

Thus it is another aspect, advantage, objective and embodiment of the present invention to teach a wearable signal acquisition and computerized display device wherein the projected display is interactive with the subject wearing the device, in that the projected display is dependent upon at least one member selected from the group consisting of: the at least one measured attribute, changes in the at least one measured attribute, changes in the at least one measured attribute with respect to time, and combinations thereof.

Thus it is another aspect, advantage, objective and embodiment of the present invention to teach a wearable signal acquisition and computerized display device further comprising a second sensor and a second signal conditioning means for sensing a physiologic variable from a second position on such subject, the second position different than the first position.

Thus it is another aspect, advantage, objective and embodiment of the present invention to teach a wearable signal acquisition and computerized display device wherein the first sensor is configured to sense a brainwave signal.

Thus it is another aspect, advantage, objective and embodiment of the present invention to teach a wearable signal acquisition and computerized display device wherein the first sensor is configured to sense a brainwave signal near such frontal lobe and the second sensor is configured to sense a brainwave signal from a more electrically-neutral position with respect to brainwaves.

Thus it is another aspect, advantage, objective and embodiment of the present invention to teach a wearable signal acquisition and computerized display device wherein the second sensor is configured to sense a brainwave signal from a position at an ear.

Thus it is another aspect, advantage, objective and embodiment of the present invention to teach a wearable signal acquisition and computerized display device wherein the device takes a difference either in software or hardware, between the brainwave signals at the first and second sensors to produce a difference brainwave signal and then processes the difference brainwave signal to produce an Attention Indicator signal indicative of the subject's intensity of focused attention, or another indicator signal indicative of one of the attributes.

Thus it is another aspect, advantage, objective and embodiment of the present invention to teach a wearable signal acquisition and computerized display device wherein the device further comprises:

a computer program cooperating with the device,

and further wherein:

the cooperating computer program takes the difference between two signals.

Thus it is another aspect, advantage, objective and embodiment of the present invention to teach a wearable signal acquisition and computerized display device wherein the Attention Indicator signal is inversely proportional to any mathematical transformation of an amplitude measure of the difference brainwave signal.

Thus it is another aspect, advantage, objective and embodiment of the present invention to teach a wearable signal acquisition and computerized display device wherein an algorithmic computation, a calculation, and a signal processing are performed within the wearable device itself.

Thus it is another aspect, advantage, objective and embodiment of the present invention to teach a wearable signal acquisition and computerized display device wherein an algorithmic computation, a calculation, and a signal processing are performed external to the device at a third location, via electronic communications over a network, this external performance having at least one characteristic selected from the group consisting of: performance at a local third location, performance at a remote third location, an analog electronic communication, a digital electronic communication, a wireless network, a wired network, and combinations thereof.

A method for training an individual having eyes and brainwaves and a surrounding real world physical environment, the method used to improve at least one of attention, focus, concentration, alertness, and mood, the method comprising:

putting onto such individual a wearable signal acquisition and computerized display device having a visual display;

interacting with the wearable signal acquisition and computerized display device to generate neurofeedback data through a projected display visible to such individual.

A method for training an individual having eyes and brainwaves and a surrounding real world physical environment, wherein the projected display is visible on lenses of the wearable signal acquisition and computerized display device, the lenses being part of an item of headgear selected from the group consisting of: glasses, goggles, a helmet, a cap, a hat, a headband, a sweatband, a spring-loaded arm mounted on the ear, other head gear and combinations thereof.

A method for training an individual having eyes and brainwaves and a surrounding real world physical environment, wherein:

such individual can still see and interact with such surrounding environment while using the wearable signal acquisition and computerized display device and simultaneously viewing the projected display.

A method for training an individual having eyes and brainwaves and a surrounding real world physical environment, for use with such individual wherein such individual is a rehabilitation patient having a relevant impaired body part, wherein:

the wearable signal acquisition and computerized display device is used to teach such rehabilitation patient to focus on such relevant impaired body part, whereby such patient receives one benefit selected from the group consisting of: body part functionality improvement, quality of treatment improvement, reduction of a duration of such rehabilitation, allowing such patient to work on such rehabilitation independently of a health care facility, allowing such patient to work on such rehabilitation independently of a health care professional, and combinations thereof.

A system for remote patient monitoring by a health professional such as a physician, therapist, counselor, coach, the system comprising:

the wearable signal acquisition and computerized display device of claim 1 and further comprising:

a transmitter for receiving data gathered by the sensor and communicating the data to such health professional.

A system for remote patient monitoring by a health professional such as a physician, therapist, counselor, coach, further comprising:

a monitor accessible to such health professional, the monitor remote from the wearable signal acquisition and computerized display device and the data communicated by means of one member selected from the group consisting of: an analog electronic communication, a digital electronic communication, a wireless network, a wired network, and combinations thereof.

### INDEX TO REFERENCE NUMERALS

Glasses embodiment 100

Front sensor 102

Rear sensors 104, 106

Goggles embodiment 200

Screen 202

Rim 204

Headband 206

Exemplary sensors 208, 210

Helmet embodiment 300

Helmet 302

Screen/faceplate 304

Sensor/electrode 306

Electronic control/memory 308

Display area 310

Electronic control/memory 400

Modules 402

Brain 404

Screen/display/heads-up unit 406

Electrode 410

Electrode lead 411

Alarm/Attention indicator/feedback/exercise 412

Scenery 414

First line of sight 416

Second line of sight 418

Optic nerve 420

Computer/CPU 500

Non-volatile computer memory 502

Alarm mechanism 503

Electrode I/O controller 504

Manual input device/joystick 505

Display I/O controller 506

Alarm control 508

Electrode differencer 510

Network connector 512

Health professional module 514

Game/exercise control 516

Signal processing 518

Data recording 520

### BRIEF DESCRIPTION OF THE DRAWINGS

FIG. 1 is an illustration of the wearable monitoring and training system for focus and/or mood according to a first embodiment in the form of glasses.

FIG. 2 is an illustration of the wearable monitoring and training system for focus and/or mood according to a second embodiment in the form of goggles.

FIG. 3 is an illustration of the wearable monitoring and training system for focus and/or mood according to a third embodiment in the form of a helmet.

FIG. 4 is a simplified block diagram of the components and interrelationships of the system and the user's nervous system.

FIG. 5 is a simplified block diagram of the modules and components of the electronic device controlling the system: these may be hardware components or software components stored in non-volatile memory on a computer device within the glasses/headwear of the invention.

### DETAILED DESCRIPTION OF THE INVENTION

Provided herein is a wearable signal acquisition and computerized display device having a heads-up display that measures an individual's focus, alertness, mood and other attributes based on variables (including physiologic and proprietary variables). One successful method of attribute measurement is based in part upon a decrease or inhibition of frontal lobe electroencephalogram (EEG) output signals. Through the heads-up display, live, real-time feedback is projected to a user subject and visible in glasses, goggles, or another headpiece. The wearable signal acquisition and computerized display device provides biofeedback, particularly brainwave biofeedback (neurofeedback). Individuals can use this neurofeedback information to train their brain to focus and to manage their mood and outlook by recognizing how various stimuli impact and influence their thoughts, focus, attention, mood, and other mental, emotional, and psychological factors.

Definitions

As used herein, the term focus is used to signify single-pointedness of concentration on a perception, thought, or image. Focus can be directed at anything and decreases with distraction. A subject can be relaxed, very alert, or in between while maintaining focus. Thus, focus is somewhat independent of alertness and is relatively easy to sustain. The duration of a focusing run is one measure of attention control. The inability to sustain focus is one indication of an attention disorder.

As used herein, the term alert or alertness is used to signify mental states resulting from mental effort or attention to events. Alertness is characterized by stimulation or excitement. Physiologic responses associated with alertness include the body summoning resources to respond to the stimulus, including tightening muscles, chest breathing, tension and/or anxiety. Unlike focus, sustaining alertness can be stressful and fatiguing. Examples of alertness are an “on the edge” feeling or the “fight or flight” reaction which involve adrenalin secretion. Alertness is related to the arousal of the reticular activating system. To develop peak performance a subject needs to build their capacity for alertness by training and yet minimize its utilization. Lowering alertness enhances calmness and relaxation.

There are prefrontal pleasure centers in the brain near the medial tip of each side of the prefrontal cortex. Electrical sensors for some types of neurofeedback can be located adjacent to them on the scalp. Improving the control of the pleasure centers with the assistance of neurofeedback can result in more feelings of happiness, satisfaction, anticipation of good events, love, gratitude, peace, and lowered stress. The brain's prefrontal pleasure centers respond to dopamine and include connections to deep brain nuclei (nucleus accumbens and ventral tegmental area) that contain dopamine. Dopamine is known to enhance the conversion of short-term memory to long-term memory. Therefore, neurofeedback training to enhance the ability to stimulate the brain's prefrontal pleasure centers is also desirable because it can accelerate the learning process and improve memory. All of these positive emotions can facilitate attention, alertness, learning, memory, and peak performance. Our use of the word “mood” is meant to include the dictionary (.com) definitions of mood (1) a state or quality of feeling at a particular time (2) a distinctive emotional quality or character, and (3) a prevailing emotional tone or general attitude.

The wearable signal acquisition and computerized display device for monitoring and training attention, focus, concentration, alertness, mood, and the like may include built-in alarms as part of the heads-up display or the computer program affiliated with it to notify a user or their doctor, therapist, counselor, or coach of certain performance thresholds. For example, indicators or alarms may go off for inferior or superior performance thresholds being realized or for sustained inferior or superior performance. The alarms may be audio, visual, vibrational, or be changes in any other medium. The indicators may also include automatically sending a text message or email to a user, their counselor, or a third party. When the user is notified of the alarms they may serve as conditioning stimuli for improving performance.

In addition to alarms, the wearable signal acquisition and computerized display device may provide other suggestions, training exercises, and games that encourage and coach the user to improve their performance and/or mood. These guides may appear as part of the projected image or information graphic in the heads-up display. They may or may not be contingent upon the level of an indicator. The games may be solitary or interactive with a counselor, coach, computer, or other known or anonymous third parties.

The wearable signal acquisition and computerized display device includes at least one sensor for measuring a physiologic variable. For example, a sensor may be mounted on glasses anywhere on the bridge or specifically between the eyes and above the nose, near the frontal lobe of the brain. Or, the sensor could be on a headband, sweatband, baseball cap, or hat. As another example, a sensor may be mounted over the ear, on an ear bud, ear stem, or other ear piece. This placement near the ear decreases electrical noise and may have other advantages, such as stability.

To further eliminate noise and/or increase the usefulness of the signal a signal conditioning means may be provided to cooperate with the sensor and enhance the accuracy of the signal it receives and/or transmits. The signal conditioning means may provide one or more stages of preamplification and filtering to isolate a high quality signal and eliminate artifacts. The signal conditioning means may be incorporated as part of the wearable signal acquisition and computerized display device and/or processors associated therewith.

The sensor can be designed to measure any suitable physiologic or other variable having demonstrated value for determining focus, concentration, attention, alertness, mood, and the like. For example, the sensor may be of the type configured to measure a brainwave signal. In accordance with a preferred embodiment, difference in two or more brainwave signals coming from different regions of the brain can be used to determine the intensity of focused attention. An electrical signal originating from a more electrically neutral location with respect to brainwaves such as the top of the ear may also form part of the difference signal.

More specifically, this differential brainwave signal method implemented by a computer program cooperating with the wearable signal acquisition and computerized display device described herein is utilized as follows:
a) Obtaining a representative frontal lobe brainwave signal from at least one first sensor in an electrically connective relation to the individual's frontal lobes
b) Obtaining a representative reference signal from at least one second sensor in an electrically connective relation to a more electrically-neutral location;
c) Subtracting the representative reference signal from the representative frontal lobe brainwave signal to produce a difference brainwave signal;
d) Processing the difference brainwave signal to produce an Attention Indicator signal indicative of the individual's intensity of focused attention;
e) Inputting the Attention Indicator signal to a device; and
f) Repeating steps a-e, as desired.
   This was patented by the applicant in U.S. Pat. No. 5,983,127.

With respect to the above method, a more electrically-neutral location with respect to brainwaves than an individual's frontal lobe may be found, for example, in the vicinity of an individual's ear. The Attention Indicator signal is inversely proportional to any mathematical transformation of an amplitude measure of the difference brainwave signal.

The signal analysis computations as outlined above can be done on the wearable device or elsewhere. For example, on a nearby device through a wired or wireless connection or remotely (including over the internet) through a digital or analog signal to a processor anywhere in the world. Similarly, the patient monitoring can also be done remotely. For example, a wireless connection or secure internet communication to a website or server of doctor, physical therapist, or counselor who can view the patient data on their tablet or laptop computer or smartphone. A doctor's tablet or laptop can be used to monitor one or several patients and to perform more advanced comparisons and statistical analysis on them. Individualized data can also be accessible by each patient having their own tablet or laptop computer or smartphone.

Each lens of the glasses, goggles, or other eyepiece of the heads-up display may be formed of any material that suitably displays a projected image or graphic. Each lens may also be sufficiently transparent to allow a user to see through the lens or a portion thereof and thereby safely interact with the real physical world while wearing the glasses or goggles. Through this combination of display and transparency features an augmented reality can be created whereby a projected image or information graphic is superimposed over a real-world view.

Different images may appear to an individual in the foreground and the background of the lenses of the glasses, goggles, helmet, headwear, or other computer display medium. Additionally, different images may be projected and assigned to the left eye and the right eye of the user.

The orientation and position of the display or user interface may be adjustable or adjust automatically depending upon movements of the user. For example, depending upon whether the user is still or in motion, and if in motion depending upon if the user is looking upward, downward, right, or left. The projected image or graphic on the visual display may not only represent mental and emotional factors including a user's concentration, focus, alertness, mood, and the like, but may also be used to show a user's interaction with an additional input device or devices (e.g. a hand remote, keyboard, joystick, or foot pedal).

### EXAMPLES OF THE INVENTION

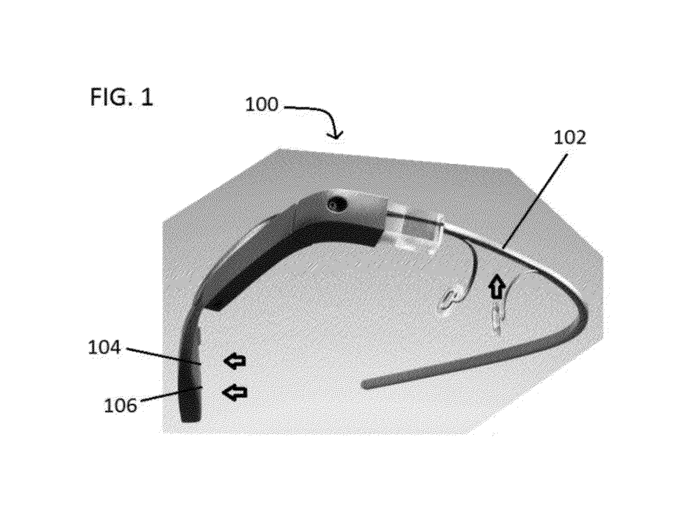

With regard to the drawings, the wearable monitoring and training system for focus and/or mood may be provided in a number of embodiments or forms in accordance with the principles of the present invention. FIG. 1 illustrates a first embodiment 100 in which the system is in the form of glasses. Electroencephalogram (EEG) sensors or other sensors may be placed at locations 102, 104, and 106. The front sensor 102 around the bridge of the glasses between the eyes and above the nose measures a signal emulating from the frontal lobe of the brain. The rear sensors 104, 106 may be placed at, near, or behind one or more of the ears at a more electrically neutral position. One of the ear sensors 106 may be an electrical ground, more properly referred to as an electrode.

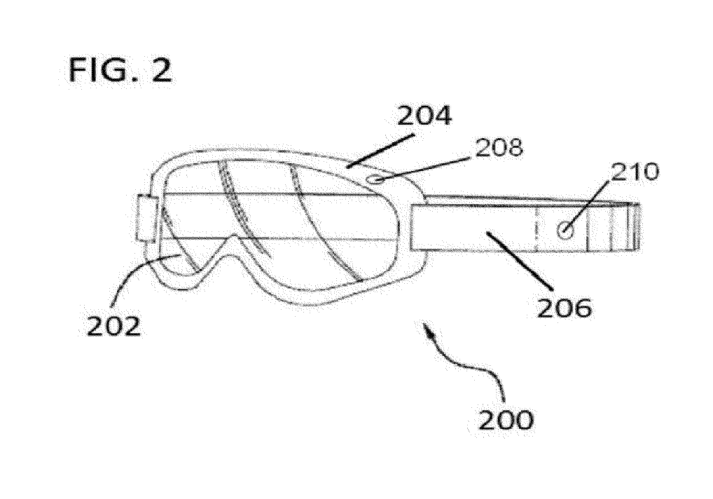

FIG. 2 shows a second embodiment 200 in which the system is in the form of goggles. Projected information may be displayed on a screen 202. Sensors may be placed along the rim 204 of the screen, along the headband 206 that holds the goggles in position and runs along the ears and back of the head, or along extensions from either the rim or the band (not shown).

Exemplary sensors are 208, 210 depicted upon the headband and within the rim 204 of the goggles. However, these locations are merely exemplary. As discussed above, there are a number of locations and more importantly, a number of combinations of locations which may be employed without departing from the scope of the invention.

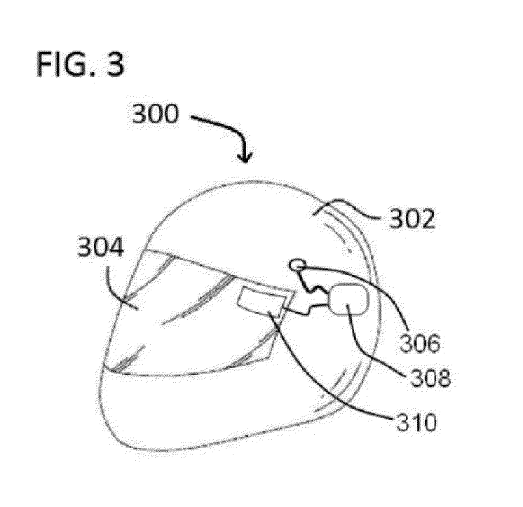

FIG. 3 shows a third embodiment 300 in which the system is in the form of a helmet. Sensors may be placed anywhere along the inside cover of the helmet 302. Projected information may be displayed on the screen 304.

Sensor/electrode 306 is shown to be individual, however, other electrodes may be invisible at other locations within the helmet. As discussed previously, in preferred embodiments there are multiple sensors/electrodes and a signal difference engine is employed to take the difference between the signals for use in the feedback loop.

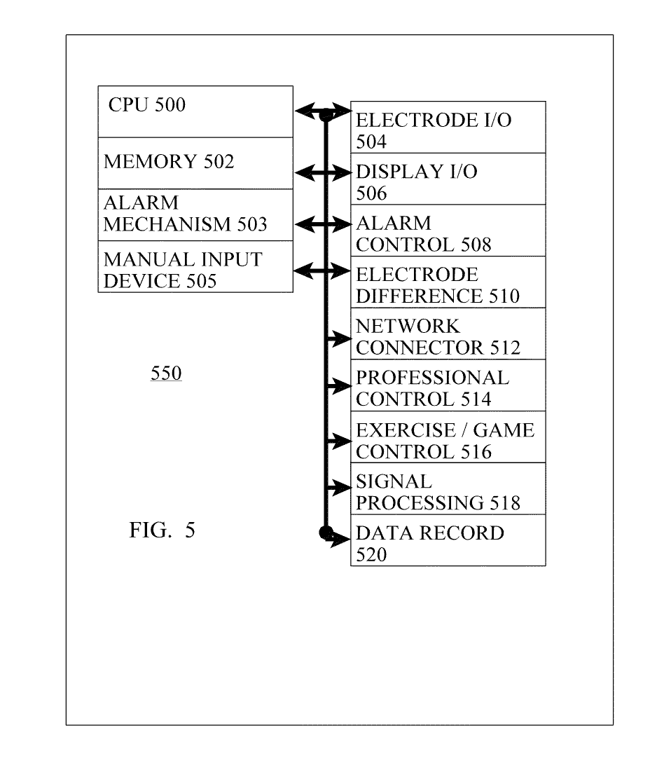

Electronic control/memory 308 is the electronic device as discussed previously. Note that it may be a protrusion or a bulge, especially on smaller devices such as the glasses embodiment, etc. The content of the electronics 308 may be found discussed further in reference to FIG. 5.

Display area 310 is shown as a rectangle on the screen, however, the entire screen may be used, or only a part thereof, in any convenient size or shape.

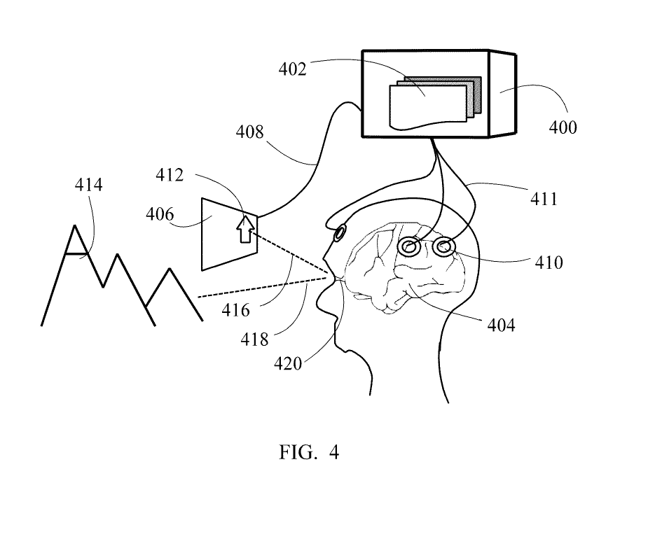

FIG. 4 is a simplified block diagram of the components and interrelationships of the system and the user's nervous system. Note that this is NOT a depiction of a physical system but rather is a block diagram showing both the system and the operational relationships of the parts of the system.

Electronic control/memory 400 contains programmed, or hardwired within it, modules 402, which are discussed in greater detail with reference to FIG. 5. The use of programmed modules reduces cost and increases flexibility, however, the use of hardwired modules produces a faster response time and more reliable operations, thus any alternative embodiment is covered within the scope of the invention and application.

Brain 404 is the user's brain, obviously, shown because it is the object of the therapy/training and further because it is a part of the feedback loop which the present invention creates and is another portion thereof.

Screen/display/heads-up unit 406 may take any form as discussed herein (glasses, a helmet, a heads-up display, etc) but in use, it is clearly visible to the user and is connected to the electronic control 400 either by wiring 408 or wirelessly.

Electrode 410 may take any form now known or later devised, one preferred embodiment thereof is contact electrodes which sense readings through the skin. Sensors may be stainless steel, other metals, sponge-covered silver-silver chloride coated ABS plastic (with saline), conducting fiber cloth and so on.

Electrode lead 411 is depicted as a wire, however, various wireless means of communication may be used in electrodes later devised.

Alarm/Attention indicator/feedback/exercise 412 is shown in this case as a simple arrow projected up on the display 406. An arrow pointing up might indicate that the subject should increase their mood or focus, or might indicate an increase in some desirable or undesirable data stream being monitored from their brain. It might be a cursor, part of an exercise, part of a game, and so on and so forth.

Scenery 414 is depicted in order to depict schematically the importance of line of sight. First line of sight 416 is from the user's eye to the indicator 412 on display 406. This would be used if the user were to make a check of their neural condition and biofeedback. On the other hand, second line of sight 418 allows the user to easily monitor the real world around them, simply by shifting their eyeball. Note that in use, it is anticipated that the device can be part of a multi-tasking regime in which the user is not restricted to only the feedback exercises, but rather also has the ability to do athletics, carry on normal daily life, drive, ride, and so on. The device and method of the invention clearly promote certain activities and thus may in fact be used in conjunction with such activities to increase performance.

Optic nerve 420 is shown merely to demonstrate that the feedback loop is a closed, complete loop, a prerequisite and part of the definition of feedback.

FIG. 5 is a simplified block diagram of the modules and components of the electronic device controlling the system: these may be hardware components or software components stored in non-volatile memory on a computer device within the glasses/headwear of the invention.

Computer/CPU 500 may have associated therewith non-volatile computer memory 502 on which electronically programmed modules of the invention may be stored, if any.

Optional alarm mechanism 503 may take the form of a buzzer, bell, beeper, voice or the like, as alarms and attention indicators may be used in several different ways in the scope of the invention: as part of the feedback loop, as an indication of problems, as a notification to health professionals, as a notification to the user, as a supplement to the visual biofeedback loop and so on and so forth.

Electrode I/O controller 504 carries out routine electronic functions necessary to interface the electronics with the physical electrodes or their leads.

Optional manual input device 505 may be any of the devices discussed previously, such as a mouse, a joystick, a hand remote, keyboard, foot pedal, a touch screen and so on. This optional device may be for the use of the user during feedback exercises for mood, focus, attention, etc, or the input device may be strictly for the use of health professionals accessing or programming the device, a combination or for other uses.

Display I/O controller 506 handles the interface of the electronics with the physical display (such as display 406 or screen 304, etc).

Alarm control 508 may control both soft alarms (visual cues, communications with networks or outside health professionals in alternative embodiments, and so on) and also the optional hard alarm (buzzer, etc) discussed in relation to aspect 503 of the invention.

Electrode difference module 510 is a mechanism, whether electrical in nature, programmed, or mechanical (and thus broadly described in the past by the terms “difference engine” or “differencer”) which as described previously may take the difference between two electrode signals, such as the difference between the signals from electrodes 102 and 104 located respectively at the front and rear of the brain. In presently preferred embodiments and the best mode now contemplated for carrying out the invention, the desire for small, flexible devices means that software programming of this module rather than other means is implicated.

Network connector 512 may be a wireless or wired module which, as discussed previously, allows the device to communicate with health professionals. Health professional module 514 is obviously a command and programming module not normally accessible to the user. This health professional module 514 is for the use of health professionals in setting up the device, in determining alarm/attention limits, determining exercises available to the user and so on.

Game/exercise control module 516 allows the user some control over the module, for example, to switch from one exercise to another depending on circumstances and so on.

Signal processing module 518 carries out the function, discussed previously, of signal conditioning and enhancement, as the signals from the human brain can be less than optimal.

Optional data recording 520 is a record of the activities and “scores” of the user, again for use by the health professional, and again possibly not accessible to the user, in the interest of maintaining an accurate record.

It will be understood that the terms “modified glasses”, “modified goggles”, “modified helmet” and so on as used herein indicate that the normal glasses, electronic display glasses, goggles, helmets, headbands, etc are modified from the norm. Firstly, this is because the glasses, etc, have the electronics, electrodes, and the feedback control mechanisms associated therewith. Secondly, note that the physical shape of the glasses, goggles, etc may change from the norm, such as the protrusion of electronics 308. In practice, it has been found that such protrusions, bulges, additions and alterations of the shape and so on are sometimes necessary in order to create the device of the invention.

It will be understood that a threshold performance metric may include reaching, or never subceeding, a positive metric; remaining below, or never exceeding, a negative performance metric; time of performance success; or a more complex performance profile requiring different degrees of success over time.

Throughout this application, various publications, patents, and/or patent applications are referenced in order to more fully describe the state of the art to which this invention pertains. The disclosures of these publications, patents, and/or patent applications are herein incorporated by reference in their entireties, and for the subject matter for which they are specifically referenced in the same or a prior sentence, to the same extent as if each independent publication, patent, and/or patent application was specifically and individually indicated to be incorporated by reference.

Methods and components are described herein. However, methods and components similar or equivalent to those described herein can be also used to obtain variations of the present invention. The materials, articles, components, methods, and examples are illustrative only and not intended to be limiting.

Although only a few embodiments have been disclosed in detail above, other embodiments are possible and the inventors intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art.

Having illustrated and described the principles of the invention in exemplary embodiments, it should be apparent to those skilled in the art that the described examples are illustrative embodiments and can be modified in arrangement and detail without departing from such principles. Techniques from any of the examples can be incorporated into one or more of any of the other examples. It is intended that the specification and examples be considered as exemplary only, with a true scope and spirit of the invention being indicated by the following claims.

What is claimed is:

1. A wearable signal acquisition and computerized display device for use in close proximity to a subject's eyes and brain, including two locations on such subject's head, the device comprising:
at least a first sensor capable of producing a first signal;
signal conditioning means;
a computerized display;
the sensor configured to measure at least one attribute from such subject when wearing the device at a first location on such subject, the attribute selected from the group consisting of: attention, focus, concentration, alertness, mood and combinations thereof.

2. The wearable signal acquisition and computerized display device of claim 1, further comprising a heads-up display.

3. The wearable signal acquisition and computerized display device of claim 1, further comprising modified glasses as part of the device.

4. The wearable signal acquisition and computerized display device of claim 1, further comprising modified goggles as part of the device.

5. The wearable signal acquisition and computerized display device of claim 1, wherein the device is in a form of a modified item of headgear, the item of headgear selected from the list consisting of: helmet, cap, hat, headband, sweatband, other head gear and combinations thereof.

6. The wearable signal acquisition and computerized display device of claim 1, further comprising at least one indicator to provide notification when the at least one measured attribute meets a first threshold performance metric, the first threshold performance metric selected from the group consisting of: positive performance, negative performance, time of performance, a performance profile, and combinations thereof.

7. The wearable signal acquisition and computerized display device of claim 1, further comprising a projected display created by the device that provides neurofeedback to the subject indicative of performance of the at least one measured attribute.

8. The wearable signal acquisition and computerized display device of claim 7, wherein the projected display is interactive with the subject wearing the device, in that the projected display is dependent upon at least one member selected from the group consisting of: the at least one measured attribute, changes in the at least one measured attribute, changes in the at least one measured attribute with respect to time, and combinations thereof.

9. The wearable signal acquisition and computerized display device of claim 1, further comprising a second sensor and a second signal conditioning means for sensing a physiologic variable from a second position on such subject, the second position different than the first position.

10. The wearable signal acquisition and computerized display device of claim 1, wherein the first sensor is configured to sense a brainwave signal.

11. The wearable signal acquisition and computerized display device of claim 9 for use with a more electrically-neutral position, with respect to brainwaves, of such subject's brain, wherein the first sensor is configured to sense a brainwave signal near such frontal lobe and the second sensor is configured to sense a brainwave signal from such more electrically-neutral position.

12. The wearable signal acquisition and computerized display device of claim 11, wherein the second sensor is configured to sense a brainwave signal from a location on an ear.

13. The wearable signal acquisition and computerized display device of claim 11, wherein the device takes a difference between the brainwave signals at the first and second sensors to produce a difference brainwave signal and then processes the difference brainwave signal to produce an Attention Indicator signal indicative of the subject's intensity of focused attention.

14. The wearable signal acquisition and computerized display device of claim 13, wherein the device further comprises:
a computer program cooperating with the device,
and further wherein:
the cooperating computer program takes the difference.

15. The wearable signal acquisition and computerized display device of claim 13, wherein the Attention Indicator signal is inversely proportional to any mathematical transformation of an amplitude measure of the difference brainwave signal.

16. The wearable signal acquisition and computerized display device of claim 1, wherein an algorithmic computation, a calculation, and a signal processing are performed within the wearable device itself.

17. The wearable signal acquisition and computerized display device of claim 1, wherein an algorithmic computation, a calculation, and a signal processing are performed external to the device at a third location, via electronic communications over a network, this external performance having at least one characteristic selected from the group consisting of: performance at a local third location, performance at a remote third location, an analog electronic communication, a digital electronic communication, a wireless network, a wired network, and combinations thereof.

18. A method for training an individual having eyes and brainwaves and a surrounding real world physical environment, the method used to improve at least one of attention, focus, concentration, alertness, and mood, the method comprising:
putting onto such individual a wearable signal acquisition and computerized display device having a visual display;
interacting with the wearable signal acquisition and computerized display device to generate neurofeedback data through a projected display visible to such individual.

19. The method of claim 18, wherein the projected display is visible on lenses of the wearable signal acquisition and computerized display device, the lenses being part of an item of headgear selected from the group consisting of: glasses, goggles, a helmet, a cap, a hat, a headband, a sweatband, a spring-loaded arm mounted on the ear, other head gear and combinations thereof.

20. The method of claim 19, wherein:
such individual can still see and interact with such surrounding environment while using the wearable signal acquisition and computerized display device and simultaneously viewing the projected display.

21. The method of claim 18, for use with such individual wherein such individual is a rehabilitation patient having a relevant impaired body part, wherein:
the wearable signal acquisition and computerized display device is used to teach such rehabilitation patient to focus on such relevant impaired body part, whereby such patient receives one benefit selected from the group consisting of: body part functionality improvement, quality of treatment improvement, reduction of a duration of such rehabilitation, allowing such patient to work on such rehabilitation independently of a health care facility, allowing such patient to work on such rehabilitation independently of a health care professional, and combinations thereof.

22. A system for remote patient monitoring by a health professional such as a physician, therapist, counselor, coach, the system comprising:
the wearable signal acquisition and computerized display device of claim 1 and further comprising:
a transmitter for receiving data gathered by the sensor and communicating the data to such health professional.

23. The system of claim 21, further comprising:
a monitor accessible to such health professional, the monitor remote from the wearable signal acquisition and computerized display device and the data communicated by means of one member selected from the group consisting of: an analog electronic communication, a digital electronic communication, a wireless network, a wired network, and combinations thereof.
